(19) United States
(12) Patent Application Publication
(10) Pub. No.: US 2017/0246362 A1
(43) Pub. Date: Aug. 31, 2017

(54) SUCTION ADAPTER DEVICE

(71) Applicant: George Crawford, Anniston, AL (US)

(72) Inventor: George Crawford, Anniston, AL (US)

(21) Appl. No.: 15/516,551

(22) PCT Filed: Oct. 1, 2015
(86) PCT No.: PCT/US2015/053461

§ 371 Date: Apr. 3, 2017

Related U.S. Application Data
(60) Provisional application No. 62/058,749, filed on Oct. 2, 2014.

(51) Int. Cl. A61M 1/00

(52) U.S. Cl. CPC A61M 1/0047; A61M 1/008; A61M 1/0041; A61C 17/0208

(57) ABSTRACT

Aspects of embodiments of the present invention disclose a suction adapter used in aspirating fluid from a particular site. The adapter device may include a nozzle configured to connect with suction tubing connected with a suction source, a drain end, structurally configured to connect the adapter with a drain tube, where the drain tube may be used to aspirate a site such as a surgical site. The adapter may also include: a channel within the adapter enabling fluid flow and a suction controlling fenestration extending from the channel to the adapter's external surface. The fenestration enables a user, by progressively opening or closing the fenestration, to vary suction within the suction adapter which, in turn, varies the flow of fluid through the adapter.

### CROSS-REFERENCE TO RELATED APPLICATIONS

This Application claims the benefit of, and is related to, the following Applicant's provisional patent application: U.S. Provisional Patent Application No. 62/058,749 titled “SUCTION DEVICE” filed Oct. 2, 2014, which is incorporated herein in its entirety.

### FIELD OF THE INVENTION

The present invention relates, in general, to a suction adapter device. Specifically, to a surgical suction adapter device that enables a user to control the amount of suction within the device.

### BACKGROUND OF THE INVENTION

Human surgery entails incisions into human tissue or organs which lead to bleeding by the patient or release of bodily fluids into a surgical area. Such a release of blood or other bodily fluids hamper the surgery as they obscure the surgeon's view of the surgical site or area and thus lengthen the surgery while also placing the patient in danger. As such, surgeons have used suction devices to aspirate blood, bodily fluids and/or semi-solid biological material away from the surgical site or area. However, these suction devices tend to not have any way to effectively vary the amount of suction applied at the surgical site. In addition, some suction devices tend to clog up with tissue and other contaminants. Further still, these suction devices, by way of their many parts, enable further clogging because of the many parts of these devices and tend to be difficult to operate during surgery.

Accordingly, there is a need for a suction device adapter that would allow for a user to vary the amount of suction coming through the device. There is also a need for a device that, while enabling user variation of suction, prevents the device from being clogged with biological particles and/or other materials during the surgery. Further still, there is a need for a suction device adapter that is simple in its construction thereby reducing or eliminating the risk of it becoming clogged with tissue and other particles or materials.

### SUMMARY OF THE INVENTION

Aspects of embodiments of the present invention contemplate a suction adapter which may include a nozzle configured to connect with suction tubing where the suction tubing may be connected with a suction source, a drain end, structurally configured to connect the adapter with a drain tube, where the drain tube may be used to aspirate a surgical site, a channel within the adapter, where the channel enables flow of fluid from the drain end through the nozzle of the adapter, and a fenestration extending from the channel to the adapter's external surface.

In an aspect of an embodiment of the present invention, the fenestration of the adapter may have multiple regions.

In an aspect of an embodiment of the present invention, the fenestration of the adapter may have first, second, third and fourth regions.

In an aspect of an embodiment of the present invention, the first region of the fenestration may be adjacent to the second region and where the second region may be adjacent to the third region and where the third region may be adjacent to the fourth region.

In an aspect of an embodiment of the present invention, the different regions may be contiguously adjacent to each other. For instance, the first region may be contiguously adjacent to the second region which may be contiguously adjacent to the third region and the third region may be contiguously adjacent to the fourth region.

In an aspect of an embodiment of the present invention, the first region of the fenestration the first region may be semi-circular in shape. In another aspect of an embodiment of the present invention, the second region may be rectangular in shape. In another aspect of an embodiment of the present invention, the fourth region may be circular in shape. In another aspect of an embodiment of the present invention, the third region may be of shape that enables it to provide a contiguous intermediate region between the second and fourth regions of the fenestration. In one aspect of an embodiment of the present invention, the third region may be any one of: trapezoidal, rectangular, or triangular in shape.

In another aspect of an embodiment of the present invention, the fenestration may enable user flow increase through the channel (or may be structurally configured to do so), enabling a user to progressively increase fluid flow through the channel where a user may do so by progressively closing the first through fourth regions of the fenestration. This may be done by having the user slide his or her finger first across the fenestration starting from the first region of the fenestration leading up to the fourth region while maintaining the closing of each region during the sliding process. It should be noted that this fluid flow increase control could also be implemented by having the user slide his or her finger the other way round—first from the fourth region and progressively to the first region.

In another aspect of an embodiment of the present invention, the fenestration may enable user flow reduction control (or may be structurally configured to do so), enabling a user to progressively decrease fluid flow through the channel where a user may do so by first closing the entire fenestration and progressively opening the fourth through first regions of the fenestration. This may be done by having the user first cover or close the entire fenestration and then slide his or her finger progressively from the fourth through the first regions of the fenestration. It should be noted that this fluid flow decrease control could also be implemented by having the user slide his or her finger the other way round—first from the first region and progressively to the fourth region.

In another aspect of an embodiment of the present invention, the walls of the fenestration may be angularly slanted in relation to the channel.

In another aspect of an embodiment of the present invention, the walls of the fenestration may be perpendicular in relation to the channel.

Additional aspects, objectives, features and advantages of the present invention will become apparent from the following description of the preferred embodiments with reference to the attached drawings.

### BRIEF DESCRIPTION OF THE DRAWINGS

FIG. 1 illustrates a plan view of the suction adapter device according to an aspect of an embodiment of the present invention.

FIG. 2 illustrates a perspective view of the suction adapter device according to an aspect of an embodiment of the present invention.

FIG. 3 illustrates a sectional view of the suction adapter device according to an aspect of an embodiment of the present invention.

FIG. 4 illustrates a detailed view of the regions of a fenestration according to an aspect of an embodiment of the present invention.

FIG. 5 illustrates a sectional view of the suction adapter device showing a detailed cross-sectional view of a fenestration section of the suction adapter device according to an aspect of an embodiment of the present invention.

FIG. 6 illustrates a sectional view of the suction adapter device showing another detailed cross-sectional view of a fenestration section of the suction adapter device according to an aspect of an embodiment of the present invention.

### DETAILED DESCRIPTION OF THE INVENTION

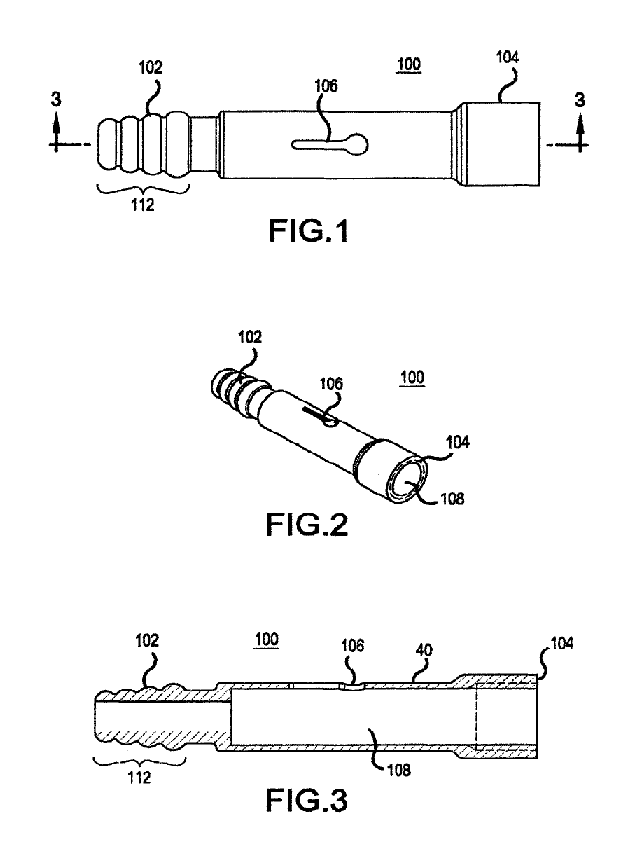

Referring now to FIGS. 1-3, different views of a suction adapter or device 100 are shown. Suction adapter 100 may include nozzle 102 configured to connect with suction tubing (not shown) where the suction tubing may be connected with a suction source (not shown). Suction adapter 100 may also include drain end 104, located at an opposite end from nozzle 102 and structurally configured to connect suction adapter 100 with a drain tube (not shown), where the drain tube may be used to aspirate a surgical site. In an aspect of an embodiment of the present invention drain end 104 may be beveled and/or curved.

Suction adapter 100 may also include channel 108 which extends lengthwise through the interior of suction adapter 100 and where channel 108 enables flow of fluid from drain end 104 as suction is applied by way of the suction source connected with nozzle 102. Control of the amount of suction is enabled by fenestration 106 extending from channel 108 to external surface 110 of suction adapter 100.

It should be noted that suction adapter 100 may be straight to facilitate its connection with either suction tubing or drain tubes. In aspects of embodiments of the present invention, suction adapter 100 may have one or more bends along its length. In aspects of embodiments of the present invention, suction adapter 100 may be any shape suitable for surgical use.

In an aspect of an embodiment of the present invention, nozzle 102 may include threads 112 configured to theadedly secure nozzle 102 to a suction tube or equivalent structure. In another aspect of an embodiment of the present invention, drain end 104 of suction adapter 100 may be either the male or female portion of a luer lock connector for connecting to a drain tube. In aspects of embodiments of the present invention, drain end 104 of suction adapter 100 may be of any connection configuration meant to enable its connection with a drain tube.

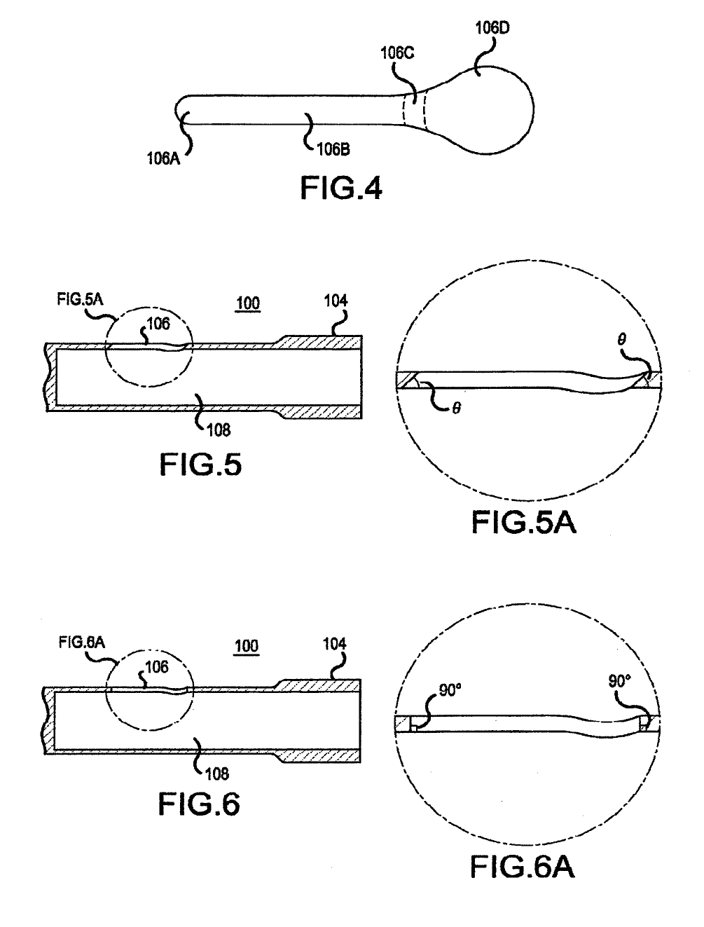

In an aspect of an embodiment of the present invention, fenestration 106 of the adapter may have multiple regions as shown in FIG. 4. In an aspect of an embodiment of the present invention, fenestration 106 of the adapter may have first region 106A, second region 106B, third region 106C and fourth region 106D.

In an aspect of an embodiment of the present invention, first region 106A of fenestration 106 may be adjacent to second region 106B where second region may be adjacent to third region 106C and where third region 106C may be adjacent to fourth region 106D.

In an aspect of an embodiment of the present invention, the different regions may be contiguously adjacent to each other. For instance, first region may be contiguously adjacent to second region 106B which may be contiguously adjacent to third region 106C and third region 106C may be contiguously adjacent to fourth region 106D.

In an aspect of an embodiment of the present invention, first region 106A of fenestration 106 may be semi-circular in shape. In another aspect of an embodiment of the present invention, second region 106B may be rectangular in shape. In another aspect of an embodiment of the present invention, fourth region 106D may be circular in shape. In another aspect of an embodiment of the present invention, third region 106C may be of shape that enables it to provide a contiguous intermediate region between the second and fourth regions of fenestration 106. In one aspect of an embodiment of the present invention, third region 106C may be any one of: trapezoidal, rectangular, or triangular in shape.

In another aspect of an embodiment of the present invention, fenestration 106 may enable user flow increase through channel 108 (or may be structurally configured to do so), enabling a user to progressively increase fluid flow through channel 108 where a user may do so by progressively closing the first through fourth regions of fenestration 106. This may be done by having the user slide his or her finger first across fenestration 106 starting from the first region of fenestration 106 leading up to fourth region 106D while maintaining the closing of each region during the sliding process. It should be noted that this fluid flow increase control could also be implemented by having the user slide his or her finger the other way round—first from fourth region 106D and progressively to the first region.

In another aspect of an embodiment of the present invention, fenestration 106 may enable user flow reduction control (or may be structurally configured to do so), enabling a user to progressively decrease fluid flow through channel 108 where a user may do so by first closing the entire fenestration and progressively opening the fourth through first regions of fenestration 106. This may be done by having the user first entirely cover or close fenestration 106 and then slide his or her finger progressively from fourth (106D) through the first region 106A of fenestration 106. It should be noted that this fluid flow decrease control could also be implemented by having the user slide his or her finger the other way round—first from first region 106A and progressively to fourth region 106D.

Referring now to FIGS. 5 and 6 sectional views of suction adapter 100 showing detailed cross-sectional views of fenestration 106 of suction adapter 100 are shown according to aspects of embodiments of the present invention. As shown in FIG. 5, the walls of fenestration 106 may be angularly slanted in relation to channel 108 forming an angle between the walls and channel 108. In another aspect of an embodiment of the present invention, the walls of fenestration 106 may be perpendicular in relation to channel 108 as shown in FIG. 6.

In another aspect of an embodiment of the present invention, suction adapter 100 may be manufactured from steel or another suitable metal or metal alloy. As such, suction adapter 100 may be reusable. In some aspects of embodiments of the present invention, suction adapter 100 may be made of plastic. In other aspects of embodiments of the present invention, components of suction adapter 100 may be made from different materials, for example, drain end 104 could be made of plastic while nozzle 102 and the rest of suction device 100 could be made of metal. In some aspects of embodiments of the present invention, suction adapter 100 may be manufactured from any suitable material or combination of materials known to one of ordinary skill in the art.

The invention has been described in detail with particular reference to certain preferred embodiments thereof, but it will be understood that variations and modifications can be effected within the spirit and scope of the invention.

What is claimed is:

1. A suction adapter comprising:
a nozzle configured to connect with suction tubing wherein the suction tubing is connected with a suction source;
a drain end, structurally configured to connect the adapter with a drain tube, wherein the drain tube is used to aspirate a surgical site;
a channel within the adapter, wherein the channel enables flow of fluid from the drain end through the nozzle of the adapter; and
a fenestration extending from the channel to the adapter's external surface.

2. The adapter of claim 1, wherein the fenestration comprises of multiple regions.

3. The adapter of claim 2, wherein the fenestration comprises of first, second, third and fourth regions.

4. The adapter of claim 3, wherein the first region is adjacent to the second region and wherein the second region is adjacent to the third region and wherein the third region is adjacent to the fourth region.

5. The adapter of claim 4 wherein the first region is semi-circular in shape and wherein the second region is rectangular in shape and wherein the fourth region is circular in shape and wherein the third region is of any one of: trapezoidal, rectangular, or triangular in shape.

6. The adapter of claim 5, wherein the fenestration is structurally configured to progressively increase fluid flow through the channel wherein a user may do so by progressively closing the first through fourth regions of the fenestration.

7. The adapter of claim 5, wherein the fenestration is structurally configured to progressively decrease fluid flow through the channel wherein a user may do so by first closing the entire fenestration and progressively opening the fourth through first regions of the fenestration.

8. The adapter of claim 1, wherein the walls of the fenestration are angularly slanted in relation to the channel.

9. The adapter of claim 1, wherein the walls of the fenestration are perpendicular in relation to the channel.
